(19) United States
(12) Patent Application Publication
(10) Pub. No.: US 2024/0181110 A1
(43) Pub. Date: Jun. 6, 2024

(54) STRIP DISPENSER FOR INDIVIDUALLY DISPENSING SMALL SINGLE-USE MEDICAL DEVICES, METHOD OF MAKING AND METHOD OF USING

(71) Applicant: 1WORLDVISTA MEDIACAL, INC., Rio Verde, AZ (US)

(72) Inventor: Michael Ferlic, Roseville, MN (US)

(73) Assignee: 1WORLDVISTA MEDIACAL, INC., Rio Verde, AZ (US)

(21) Appl. No.: 18/554,945

(22) PCT Filed: Apr. 1, 2022
(86) PCT No.: PCT/US2022/071505

§ 371 Date: Oct. 11, 2023

Related U.S. Application Data
(60) Provisional application No. 63/173,516, filed on Apr. 12, 2021.

(51) Int. Cl. A61L 2/26; A61L 2/18

(52) U.S. Cl. CPC A61L 2/26; A61L 2/18; A61L 2202/16; A61L 2202/182; A61L 2202/24

(57) ABSTRACT

A strip dispenser (10) for individually dispensing small single-use medical devices (30) retained within a housing (40), method of making the dispenser (10) and method of using the dispenser (10). The housings (40) are individually detachably attached along the lateral edges (20y) of a common retention implement 20 in a longitudinally x spaced arrangement.

### BACKGROUND

Medical end-site disinfecting caps (hereinafter “disinfecting caps’) have become a ubiquitous tool used throughout the world to improve patient care by disinfecting medical end-sites such as I.V. needleless valve connectors prior to use. Exemplary disinfecting caps are described in U.S. Pat. Nos. 4,440,207, 7,282,186, 7,780,794, 8,162,899, 8,252,247, 8,273,303, 8,388,894, 8,523,831, 8,808,637, 8,828,327, 9,561,298, 9,572,904, 9,707,308 and US Patent Application Publications 2007/0112333, 2009/0137969, 2017/0157386 and 2017/0312377, each of which is incorporated herein by reference. Generally, each disinfecting cap comprises a disinfecting element—configured and arranged for attachment to a medical end-site, retained along with a supply of a disinfectant such isopropyl alcohol within a housing and hermetically sealed with a peel off lid.

One commercially available disinfecting cap is offered by 3M under the brand Curos™. The Curos™ disinfecting caps are provided as a linear array of several caps sealingly attached to a common longitudinally elongated flexible strip which functions as the peel off lid. The strip has an orifice at one end for suspension from an I.V. pole. Each disinfecting cap is individually peelable from the strip. This dispensing technique suffers several drawbacks including the limited number of disinfecting caps which can be provided on each strip before the length of the strip becomes unwieldy.

Another commercially available disinfecting cap is offered by Saxa Medical Solutions, Inc. under the brands DOCit™ and Hubscrub™. Each DOCit™ and Hubscrub™ disinfecting cap is provided as a separate and individual unit. Saxa offers a workstation for holding a supply of the DOCit™ and Hubscrub™ disinfecting caps, described in detail in Int'l Patent Application Publication WO 2018/201122 A1. The workstation technique for storing and dispensing the disinfecting caps also suffers several drawbacks including the relatively high cost and cumbersome nature of the workstation.

Accordingly, a substantial need exists for an inexpensive improved dispenser for dispensing small single-use medical devices such as disinfecting caps.

### SUMMARY OF THE INVENTION

A first aspect of the invention is a strip dispenser for individually dispensing small single-use medical devices. The dispenser includes a longitudinally elongated retention implement and a plurality of appendant small single-use medical devices. The retention implement has first and second longitudinally spaced ends, first and second laterally spaced edges and first and second transversely spaced surfaces. Each small single-use medical device comprises a housing which is a component of or contains the small single-use medical device and has an access opening through the housing. The access opening is sealed with a removable lid. The housings are individually detachably attached to and laterally extend from at least one of the first and second laterally spaced edges of the retention implement in a longitudinally spaced arrangement.

In a first particular embodiment of the strip dispenser, the retention implement includes at least two laminated material layers with each housing individually detachably attached to the retention implement by positioning a minor portion of each housing between the laminated material layers and securing this minor portion of each housing to the retention implement.

In a second particular embodiment of the strip dispenser, the retention implement includes at least two laminated material layers and each housing is individually detachably attached to the retention implement by positioning a minor portion of each lid between the laminated material layers and securing this minor portion of each lid to the retention implement.

A second aspect of the invention is a refillable receptacle for dispensing presentation of a strip dispenser according to the first aspect of the invention. The receptacle includes a means for suspending the receptacle from a point of attachment on an IV pole and a rigid frame configured and arranged to releasably capture the retention implement without hindering detachability of each individual housing from the retention implement.

A third aspect of the invention is a method of dispensing small single-use medical devices from the strip dispenser according to the first aspect of the invention.

In a first embodiment, the method of dispensing includes the steps of (A) hanging the strip dispenser from a point of attachment whereby a longitudinal axis of the strip dispenser extends substantially in a vertical direction, (B) pinching one of the plurality of housings and associate lid, and (C) pulling the pinched housing and associate lid away from the point of attachment until the pinched housing and associate lid detach from the retention implement.

In a second embodiment, the method of dispensing includes the steps of (A) hanging the strip dispenser from a point of attachment whereby a lateral axis of the strip dispenser extends substantially in a vertical direction, (B) pinching one of the plurality of housings and associate lid, and (C) pulling the pinched housing and associate lid away from the point of attachment until the pinched housing and associate lid detach from the retention implement.

In a third embodiment, the method of dispensing includes the steps of (A) hanging the strip dispenser from a point of attachment whereby a longitudinal axis of the strip dispenser extends substantially in a vertical direction, (B) pinching one of the plurality of housings without pinching the associate lid, and (C) pulling the pinched housing away from the point of attachment until the pinched housing peels off from the lid.

In a fourth embodiment, the method of dispensing includes the steps of (A) hanging the strip dispenser from a point of attachment whereby a lateral axis of the strip dispenser extends substantially in a vertical direction, (B) pinching one of the plurality of housings without pinching the associate lid, and (C) pulling the pinched housing away from the point of attachment until the pinched housing peels off from the lid.

In a fifth embodiment, the method of dispensing includes the steps of (A) hanging a strip dispenser in accordance with the first particular embodiment of the strip dispenser from a point of attachment, (B) peeling the first material layer of the retention implement away from the second material layer of the retention implement past the minor portion of a first housing so as to uncover the minor portion of the first housing, (C) detaching the first housing and associated lid from the retention implement, and (D) repeating steps (B) and (C) for sequential housings on the strip dispenser.

In a sixth embodiment, the method of dispensing includes the steps of (A) hanging a strip dispenser in accordance with the second particular embodiment of the strip dispenser from a point of attachment, (B) peeling the first material layer of the retention implement away from the second material layer of the retention implement past the minor portion of a first lid so as to uncover the minor portion of the first lid, (C) detaching the first lid and associated housing from the retention implement, and (D) repeating steps (B) and (C) for sequential housings on the strip dispenser.

A fourth aspect of the invention is a method of manufacturing a strip dispenser for individually dispensing small single-use medical devices.

A first embodiment of the method of manufacturing includes the steps of (A) obtaining a first material layer and a second material layer, each having first and second longitudinally spaced ends, first and second laterally spaced edges and first and second transversely spaced surfaces, (B) obtaining a plurality of housings, each comprising a component of or containing the same small single-use medical device and having an access opening through the housing sealed with a removable lid, (C) positioning a plurality of the housings in longitudinally spaced relationship to one another along at least one of the edges of the first material layer with a minor portion of each housing overlapping the first surface of the first material layer, (D) superimposing the second material layer over the first material layer with the first surface of the second material layer facing the first surface of the first material layer so as to sandwich the minor portion of each housing overlapping the first surface of the first material layer between the first material layer and the second material layer, and (E) bonding the first and second material layers together to form a retention implement with the minor portion of each housing sandwiched between the first and second material layers detachably secured to the retention implement.

A second embodiment of the method of manufacturing includes the steps of (A) obtaining a first material layer and a second material layer, each having first and second longitudinally spaced ends, first and second laterally spaced edges and first and second transversely spaced surfaces, (B) obtaining a plurality of housings, each comprising a component of or containing the same small single-use medical device and having an access opening through the housing sealed with a removable lid, (C) positioning a plurality of the housings in longitudinally spaced relationship to one another along at least one of the edges of the first material layer with a minor portion of each lid overlapping the first surface of the first material layer, (D) superimposing the second material layer over the first material layer with the first surface of the second material layer facing the first surface of the first material layer so as to sandwich the minor portion of each lid overlapping the first surface of the first material layer between the first material layer and the second material layer, and (E) bonding the first and second material layers together to form a retention implement with the minor portion of each housing sandwiched between the first and second material layers detachably secured to the retention implement.

### BRIEF DESCRIPTION OF THE DRAWINGS

FIG. 1 is a top view of one embodiment of the invention.

FIG. 2 is a bottom view of the invention depicted in FIG. 1.

FIG. 3 is a side view of the invention depicted in FIG. 1.

FIG. 4 is a cross-sectional view of one embodiment of a small single-use medical device suitable for attachment to and dispensing from the invention.

### DETAILED DESCRIPTION OF A PREFERRED EMBODIMENT

### Definitions

As utilized herein, including the claims, “small”, when used in reference to a medical device means a volume of less than 30 cm³ exclusive of any packaging typically separated from the medical device and discarded prior to use of the medical device.

As utilized herein, including the claims, the phrase “substantially in a vertical direction” means within 20° of vertical.

As utilized herein, including the claims, the terms “vertical” and “horizontal” are in reference to the direction of earth imposed gravity, with vertical running parallel to the direction of gravity and horizontal running perpendicular to the direction of gravity.

Nomenclature

| REFERENCE | |
|---|---|
| NO. | NAME |
| 10 | Strip Dispenser |
| 10x | Longitudinal Axis of Strip Dispenser |
| 20 | Retention Implement |
| 20m¹ | First Lateral Side Margin of Retention Implement |
| 20m² | Second Lateral Side Margin of Retention Implement |
| 20s | Spine Portion of Retention Implement |
| 20x_{Long} | Longitudinal Length of Retention Implement |
| 20x¹ | First Longitudinal End of Retention Implement |
| 20x² | Second Longitudinal End of Retention Implement |
| 20y_{Wide} | Lateral Width of Retention Implement |
| 20y¹ | First Lateral Edge of Retention Implement |
| 20y² | Second Lateral Edge of Retention Implement |
| 20z_{Thick} | Transverse Thickness of Retention Implement |
| 20z¹ | First Transverse Surface of Retention Implement |
| 20z² | Second Transverse Surface of Retention Implement |
| 21 | First Material Layer of Retention Implement |
| 21x¹ | First Longitudinal End of First Material Layer of Retention Implement |
| 21x² | Second Longitudinal End of First Material Layer of Retention Implement |
| 21y¹ | First Lateral Edge of First Material Layer of Retention Implement |
| 21y² | Second Lateral Edge of First Material Layer of Retention Implement |
| 21z¹ | First Transverse Surface of First Material Layer of Retention Implement |
| 21z² | Second Transverse Surface of First Material Layer of Retention Implement |
| 22 | Second Material Layer of Retention Implement |
| 22x¹ | First Longitudinal End of Second Material Layer of Retention Implement |
| 22x² | Second Longitudinal End of Second Material Layer of Retention Implement |
| 22y¹ | First Lateral Edge of Second Material Layer of Retention Implement |
| 22y² | Second Lateral Edge of Second Material Layer of Retention Implement |
| 22z¹ | First Transverse Surface of Second Material Layer of Retention Implement |
| 22z² | Second Transverse Surface of Second Material Layer of Retention Implement |
| 25 | Tabs |
| 28 | Line of Lamination |
| 29 | Orifice Through the Retention Implement |
| 30 | Medical Device |
| 40 | Housing |
| 40a | Enclosure Portion of Housing |
| 49 | Access Opening Through Housing |
| 50 | Functional Element |
| 60 | Disinfectant |
| 70 | Lid |
| 70a | Sealing Portion of Lid |
| 110 | Line of Weakness |
| 120 | Area of Attachment |
| 125 | Relief Pattern of Raised Protuberances |
| x | Longitudinal Axis |
| y | Lateral Axis |
| x | Transverse Axis |

### Construction

### Common Structure

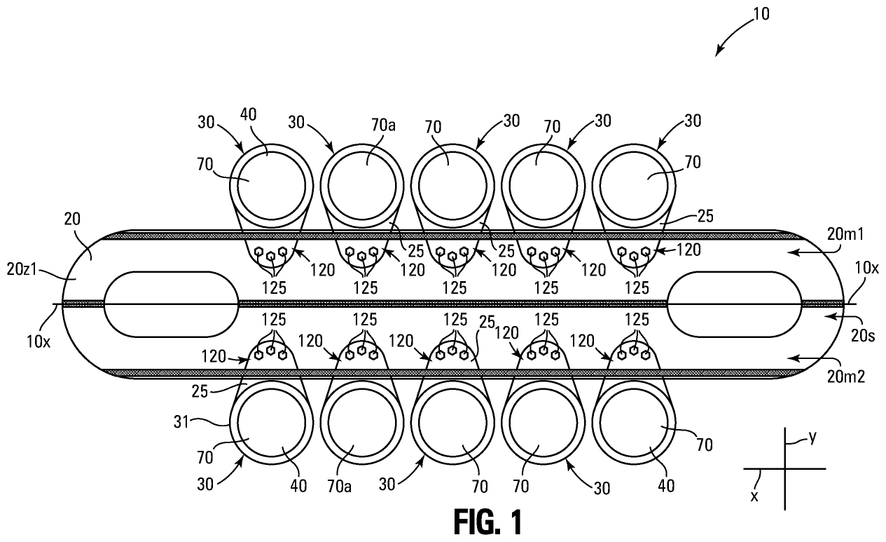

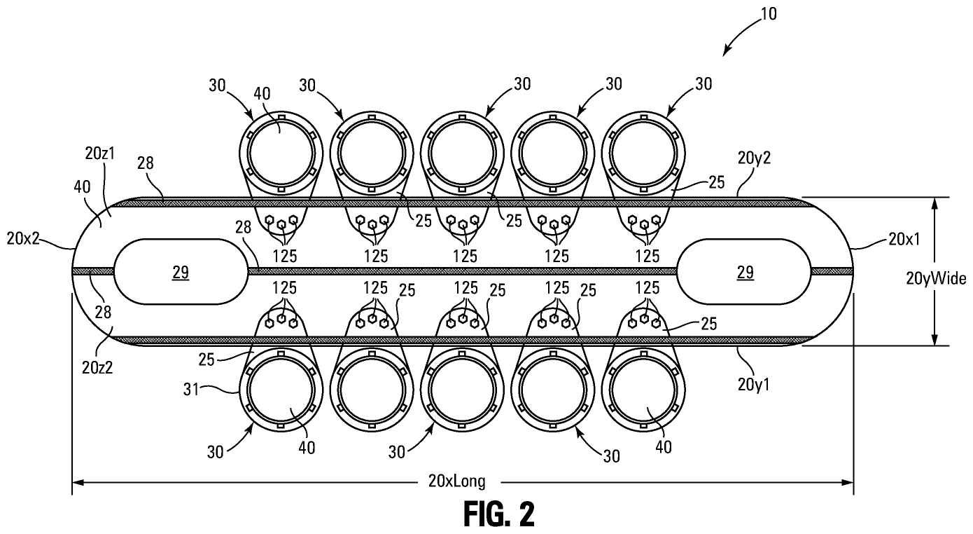

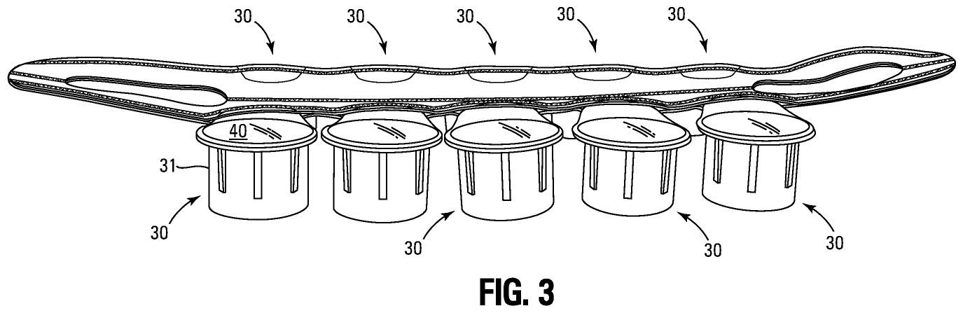

Referring to FIGS. 1-4, the strip dispenser 10 includes a plurality of small single-use medical devices 30 detachably extending from at least one and preferably both lateral edges 20y¹ and 20y² of a retention implement 20. The strip dispenser 10 preferably includes at least six medical devices 30 appending from the two lateral edges 20y¹ and 20y² of the retention implement 20 in laterally y aligned pairs or a longitudinally offset zipper pattern. The small single-use medical devices 30 can be the same or different single-use medical devices 30. The strip dispenser 10 defines a longitudinal axis 10x and is preferably symmetrical about the longitudinal axis 10x.

The retention implement 20 has first and second longitudinal ends 20x¹ and 20x² defining a longitudinal length 20x_{Long}, first and second lateral edges 20y¹ and 20y² defining a lateral width 20y_{Wide}, and first and second transverse surfaces 20z¹ and 20z² defining a transverse thickness 20z_{Thick}. Generally suitable dimensions are set forth in Table One below.

TABLE ONE

| | | | | | MOST | |
|---|---|---|---|---|---|---|
| DIMENSION | SUITABLE | | PREFERRED | | PREFERRED | |
| Longitudinal Length | 3-12 | inches | 3-8 | inches | 4-6 | inches |
| (20x_{Long}) | | | | | | |
| Lateral Width | 0.5-4 | inches | 0.5-3 | inches | 0.5-1 | inches |
| (20y_{Wide}) | | | | | | |
| Transverse | <500 | mil | <100 | mil | <50 | mil |

| | | | |
|---|---|---|---|
| Thickness (20z_{Thick}) | | | |
| Aspect Ratio | 2:1 to 20:1 | 3:1 to 10:1 | 4:1 to 8:1 |
| Longitudinal Length | | | |
| to Lateral Width | | | |

A retention implement 20 having a longitudinal length 20x_{Long} of less than about 3 inches unnecessarily limits the number of medical devices 30 which may be held and dispensed by the retention implement 20, while a longitudinal length 20x_{Long} of greater than about 12 inches results in a cumbersome and unwieldy strip dispenser 10.

A retention implement 20 having a lateral width 20y_{Wide} of less than about 0.5 inches complicates attachment and individual dispensing of the medical devices 30, while a lateral width 20y_{Wide} of greater than about 4 inches adds unnecessary size, bulk and expense to the strip dispenser 10.

A retention implement 20 having a transverse thickness 20z_{Thick} of less than about 4 mil is susceptible to ripping and tearing during dispensing of the medical devices 30, while a transverse thickness 20z_{Thick} of greater than about 500 mil (½ inch) adds unnecessary size, bulk and expense to the strip dispenser 10.

The retention implement 20 is preferably designed as a single use disposable item with a preference for inexpensive and ecologically friendly materials. Suitable materials include specifically but not exclusively plastic sheets, plastic film, paperboard, cardboard, Monopan™ panels, and laminates thereof. The retention implement 20 can be flexible or rigid but should possess sufficient structural integrity and rigidity to present the medical devices 30 in a planar fashion when the strip dispenser 10 is suspended from one end 20x¹ or 20x² of the retention implement 20, and avoid any unplanned ripping or tearing of the retention implement 20 as the medical devices 30 are detached therefrom.

The retention implement 20 can be conveniently conceptually partitioned into a longitudinally elongated central spine portion 20s positioned between first and second longitudinally elongated lateral side margins 20m¹ and 20m² respectively, with the medical devices 30 attached to and laterally y extend from at least one and preferably both lateral side margins 20m¹ and 20m².

The retention implement 20 preferably includes an orifice 29 proximate at least one and preferably both longitudinal ends 20x¹ and 20x² for use in suspending the strip dispenser 10 from a projection such as a peg or hook on an I.V. pole. Orifices 29 can also or alternatively be provided at different locations on the retention implement 20 to provide different angles of suspension.

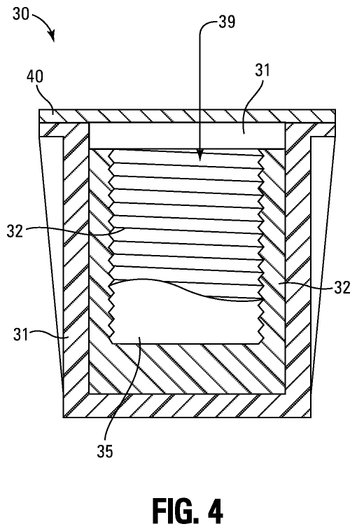

The medical devices 30 each include a housing 40 as a component of the medical device 30 or as a container for the medical device 30. Each housing 40 has an access opening 49 sealed with a removable lid 70. The medical devices 30 attached to each retention implement 20 are preferably the same small single-use medical device 30. An exemplary medical device 30 is a medical end-site disinfecting device, such as disclosed in U.S. Pat. Nos. 4,440,207, 7,282,186, 7,780,794, 8,162,899, 8,252,247, 8,273,303, 8,388,894, 8,523,831, 8,808,637, 8,828,327, 9,561,298, 9,572,904, 9,707,308 and US Patent Application Publications 2007/0112333, 2009/0137969, 2017/0157386 and 2017/0312377, incorporated herein by reference. Referring to FIG. 4, these medical end-site disinfecting devices generally include a functional element 50—configured and arranged for engagement with a medical end-site, retained along with a supply of a disinfectant 60, such as isopropyl alcohol, within a housing 40 and hermetically sealed with a peel off lid 70.

### Lid Tab

Referring to FIGS. 1-3, each housing 40 can be hermetically sealed with a lid 70 wherein a sealing portion 70a of the lid 70 is engaged to the housing 40 and sealingly covers the access opening 49 in the housing 40, and a tab portion 25 of each lid 70 is releasably secured to one of the lateral side margins 20m¹ and 20m² of the retention implement 20.

A first embodiment of this version of the invention allows each housing 40 to be peeled off its associated sealing portion 70a of the lid 70 to provide immediate access to the medical device 30 retained with the housing 40 and leaving the entire lid 70 attached to the retention implement 20. This requires a bond strength between the tab portion 25 of the lid 70 and the retention implement 20 that sufficiently exceeds the bond strength between the sealing portion 70a of the lid 70 and the housing 40 so that pulling of the housing 40 away from the lid 70 effects detachment of the housing 40 from the sealing portion 70a of the lid 70 before the tab portion 25 of the lid 70 detaches from the retention implement 20.

A second embodiment of this version of the invention provides a line of demarcation constituting a line of weakness 110, such as a line of perforations or an area of frangible material, in the lid 70 between the tab portion 25 and the sealing portion 70a of the lid 70 for facilitating individual detachment of each sealing portion 70a with an associated housing 40 still hermetically engaged to the sealing portion 70a from the tab portion 25, with the tab portion 25 remaining attached to the retention implement 20. The line of weakness 110 can be positioned along the lid 70 so as to either (i) preserve the hermetically sealed integrity of the enclosure portion 40a of the housing 40 upon detachment of the housing 40 from the retention implement 20 along the line of weakness 110, or (ii) purposely effect an opening of the enclosure portion 40a of the housing 40 sufficient to allow immediate use of the medical device 30 retained within the housing 40 upon detachment of the housing 40 from the retention implement 20 along the line of weakness 110. This requires either (i) that a user pinch or grip both the sealing portion 70a of the lid 70 and the housing 40 to prevent detachment of the housing 40 from the sealing portion 70a of the lid 70 before the sealing portion 70a of the lid 70 detaches from the tab portion 25 of the lid 70 along the line of weakness 110, or (ii) a line of weakness 110 with a tear strength sufficiently less than the bond strength between the sealing portion 70a of the lid 70 and the housing 40 so that pulling of the housing 40 away from retention implement 20 always effects detachment of the sealing portion 70a of the lid 70 from the tab portion 25 of the lid 70 along the line of weakness 110 before the housing 40 detaches from the sealing portion 70a of the lid 70.

When a line of weakness 110 is provided in the lid 70 for separating each housing 40 from the retention implement 20, the lids 70 on the same edge of the retention implement 20 can share a common tab portion 25 so long as the line of weakness 110 allows detachment of individual sections of the common tab portion 25 (i.e., individual lids 70) to be detached for each individual housing 40.

In a preferred embodiment, the retention implement 20 is comprised of at least two superimposed laminated material layers, a first material layer 21 and a second material layer 22, with the tab portion 25 of the lids 70 secured to the retention implement 20 between the material layers 21 and 22. The first and second material layers 21 and 22 may be laminated together by any expedient means including specifically but not exclusively adhesively bonded, heat welded, mechanically riveted, mechanically stapled, etc.

The tab portion 25 of the lids 70 can be secured to the retention implement 20 between the material layers 21 and 22 by embossing the overlapped tab portion 25 and material layers 21 and 22 with nested transversely z projecting relief patterns 125, such as a pattern of dimples, indents, detents, cross-hatches, raised diamonds, etc., whereby the nested relief pattern 125 is operable for resisting longitudinal x and lateral y withdrawal of the tab portion 25 of each lid 70 from between the first and second material layers 21 and 22. Alternatively, the material layers 21 and 22 can be constructed from a thermoplastic and heat welded to the tab portion 25 positioned between the material layers 21 and 22.

### Housing Tab

In yet another alternative, each housing 40 can be hermetically sealed with an individual lid 70 with an enclosure portion 40a of each housing 40 enclosing the functional element 50 and a tab portion 25 of each housing 40 secured to one of the lateral side margins 20m¹ and 20m² of the retention implement 20.

A first embodiment of this version of the invention allows each housing 40 to be individually detached from the retention implement 20 while still hermetically sealed by the lid 70 by pulling the housing 40 away from the retention implement 20 with sufficient force to overcome the bond between the tab portion 25 of each housing 40 and the retention implement 20. This requires a retention implement 20 and a housing 40 with a tear strength sufficiently greater than the bond strength between the retention implement 20 and the tab portion 25 of each housing 40 so that pulling of the housing 40 away from the retention implement 20 effects detachment of the housing 40 from the retention implement 20 before either of the retention implement 20 or the housing 40 tears or rips.

A second embodiment of this version of the invention provides a line of demarcation constituting a line of weakness 110, such as a line of perforations or an area of frangible material, in the housing 40 between the tab portion 25 and the enclosure portion 40a of the housing 40 for facilitating individual detachment of each enclosure portion 40a of the housing 40, still hermetically sealed by the lid 70, from the tab portion 25, with the tab portion 25 remaining attached to the retention implement 20. The line of weakness 110 can be positioned on the housing 40 so as to either (i) preserve the hermetically sealed integrity of the enclosure portion 40a of the housing 40 upon detachment of the housing 40 from the retention implement 20 along the line of weakness 110, or (ii) purposely effect an opening of the enclosure portion 40a of the housing 40 sufficient to allow immediate use of the medical device 30 retained within the housing 40 upon detachment of the housing 40 from the retention implement 20 along the line of weakness 110. This requires a retention implement 20 and a housing 40 with a tear strength sufficiently greater than the tear strength of the line of weakness 110 so that pulling of the housing 40 away from the retention implement 20 effects detachment of the tab portion 25 of the housing 40 from the enclosure portion 40a of the housing 40 before either of the retention implement 20 or the housing 40 tears or rips.

In a preferred embodiment, the retention implement 20 is comprised of at least two superimposed laminated material layers, a first material layer 21 and a second material layer 22, with the tab portion 25 of each housing 40 secured to the retention implement 20 between the material layers 21 and 22. The first and second material layers 21 and 22 may be laminated together by any expedient means including specifically but not exclusively adhesively bonded, heat welded, mechanically riveted, mechanically stapled, etc.

The tab portion 25 of each housing 40 can be secured to the retention implement 20 between the material layers 21 and 22 by embossing the overlapped tab portion 25 and material layers 21 and 22 with nested transversely z projecting relief patterns 125, such as a pattern of dimples, indents, detents, cross-hatches, raised diamonds, etc., whereby the nested relief pattern 125 is operable for resisting longitudinal x and lateral y withdrawal of the tab portion 25 of each housing 40 from between the first and second material layers 21 and 22. Alternatively, the material layers 21 and 22 can be constructed from a thermoplastic and heat welded to the tab portion 25 positioned between the material layers 21 and 22.

### Retention Implement Tab

Alternatively, the retention implement 20 can be formed with a plurality of tabs 25 monolithically formed with and extending laterally y from at least one lateral edge and preferably both lateral edges 20y¹ and 20y² of the retention implement 20. Each tab 25 can be hermetically sealed to a housing 40 over the access opening 49 so as to function as the lid 70.

A first embodiment of this version of the invention allows each housing 40 to be peeled off its associated tab 25 to provide immediate access to the medical device 30 retained with the housing 40 and leaving the tab 25 on the retention implement 20. This requires a retention implement 20 with a tear strength greater than the bond strength between each tab 25 and housing 40 so that pulling of the housing 40 away from the retention implement 20 effects detachment of the housing 40 from the tab 25 before the tab 25 tears or rips off the retention implement 20.

A second embodiment of this version of the invention provides a line of demarcation constituting a line of weakness 110, such as a line of perforations or an area of frangible material, in the retention implement 20 between each tab 25 and the spine portion 20s of the retention implement 20 for facilitating individual detachment of each tab 25 from the retention implement 20 with an associated housing 40 still hermetically engaged to the tab 25. This requires either (i) that a user pinch or grip both the tab 25 and the housing 40 to prevent detachment of the housing 40 from the tab 25 before the tab 25 detaches from the balance of the retention implement 20 along the line of weakness 110, or (ii) a line of weakness 110 with a tear strength sufficiently less than the bond strength between the tab 25 and the housing 40 so that pulling of the housing 40 away from retention implement 20 always effects detachment of the tab 25 from the balance of the retention implement 20 along the line of weakness 110 before the housing 40 detaches from the tab 25.

### Method of Manufacturing

One method of manufacturing the strip dispenser 10 includes the steps of (A) obtaining a first material layer 21 and a second material layer 22, each having first and second longitudinally x spaced ends 21x¹, 22x¹ and 21x², 22x² respectively, first and second laterally spaced edges 21y¹, 22y¹ and 21y², 22y² respectively, and first and second transversely z spaced surfaces 21z¹, 22z¹ and 21z², 22z² respectively, (B) obtaining a plurality of housings 40, each comprising a component of or containing the same small single-use medical device 30 and having an access opening 49 through the housing 40 sealed with a removable lid 70, (C) positioning a plurality of the housings 40 in longitudinally x spaced relationship to one another along at least one of the edges 21y¹ or 21y² of the first material layer 21 with a tab portion 25 of each housing 40 or a tab portion 25 of each lid 70 overlapping the first transverse surface 21z¹ of the first material layer 21, (D) superimposing the second material layer 22 over the first material layer 21 with the first transverse surface 22zł of the second material layer 22 facing the first transverse surface 21zł of the first material layer 21 so as to sandwich the tab portion 25 of each housing or tab portion 25 of each lid 70 overlapping the first transverse surface 21z¹ of the first material layer 21 between the first material layer 21 and the second material layer 22, and (E) bonding the first and second material layers 21 and 22 together to form a retention implement 20 with the tab portion 25 of each housing 40 or tab portion 25 of each lid 70 sandwiched between the first and second material layers 21 and 22 detachably secured to the retention implement 20.

Lines of weakness 110 can be formed in the retention implement 20, housings 40 or lids 70 for facilitating detachment of individual housings 40 from the retention implement 20 along each line of weakness 110.

The first and second material layers 21 and 22 may be bonded together by any expedient means including specifically but not exclusively adhesively bonded, heat welded, mechanically riveted, mechanically stapled, etc. The bonding may be continuous or patterned with a preference for discontinuous longitudinal lines of lamination 28 of the first and second material layers 21 and 22 occurring only between adjacent areas of attachment 120 (i.e., the tab portions 25 of either the housings 40 or the lids 70 positioned between the material layers 21 and 22).

In similar fashion, at least one and preferably both of the first and second material layers 21 and 22 is bonded to the tab portions 25 of either the housings 40 or the lids 70 positioned between the material layers 21 and 22 (hereinafter collectively area of attachment 120), with a preference for a longitudinally x continuous line of bonding of at least one of the material layers 21 and 22 to each and every tab portion 25 of either the housing 40 or the lid 70 extending from one of the lateral edges 20y¹, 20y² of the retention implement 20.

### Method of Using

Single-use medical devices 30 can be conveniently dispensed from certain embodiments of the strip dispenser 10 with the housing 40 still hermetically sealed by the lid 70 by (i) hanging the strip dispenser 10 from a point of attachment, such as a hook on an I.V. pole, with either the longitudinal axis x or the lateral axis y of the strip dispenser 10 extending substantially vertically, (ii) pinching one of the plurality of housings 40, either by pinching only the housing 40 (i.e., pinching from opposite sides of the housing 40) or both the housing and its associate lid 70 (i.e., pinching from the top and bottom of the housing 40) and then (iii) pulling the pinched housing 40 away from the point of attachment until the pinched housing 40 and associate lid 70 detach from the retention implement 20.

Single-use medical devices 30 can be conveniently dispensed from other embodiments of the strip dispenser 10 with the lid 70 removed from housing 40 during dispensing by (i) hanging the strip dispenser 10 from a point of attachment, such as a hook on an I.V. pole, with either the longitudinal axis x or the lateral axis y of the strip dispenser 10 extending substantially vertically, (ii) pinching one of the plurality of housings 40 without pinching its associate lid 70 (i.e., pinching the sides of the housing 40) and then (iii) pulling the pinched housing 40 away from the point of attachment until the pinched housing 40 peels from the associate lid 70, leaving the lid 70 attached to the retention implement 20.

When the retention implement 20 is comprised of superimposed laminated first and second material layers 21 and 22, with a tab portion 25 of either the housing 40 or the lid 70 secured to the retention implement 20 between the material layers 21 and 22 (hereinafter collectively area of attachment 120), medical devices 30 attached to the retention implement 20 can be conveniently dispensed in certain embodiments by (i) hanging the strip dispenser 10 from a point of attachment, (ii) peeling the first material layer 21 of the retention implement 20 away from the second material layer 22 of the retention implement 20 past a first area of attachment 120 and optionally a plurality of areas of attachment 120 so as to uncover and expose at least the first area of attachment 120 and optionally a plurality of areas of attachment 120, (iii) detaching the housing 40 and lid 70 attached to the retention implement 20 at each of the exposed areas of attachment 120, and (iv) repeating steps (ii) and (iii) for sequential areas of attachment 120 on the strip dispenser 10.

Additionally, the strip dispenser 10 can be held by a clinician in one hand and a housing 40 without or without its associate lid 70 detached with the other hand, in lieu of hanging or suspending the strip dispenser 10 from an I.V. pole or attaching the strip dispenser 10 to a surface mounted fixture. It can be appreciated that such manual holding of the strip dispenser 10 and removal of medical devices 30 therefrom will be the preferred method of using the strip dispenser 10 in many settings outside of a health care facility, particular home healthcare settings, where an I.V. pole or surface mounting fixture may not be present nor available.

1. A strip dispenser for individually dispensing small single-use medical devices, comprising:
a longitudinally elongated retention implement having first and second longitudinally spaced ends, first and second laterally spaced edges and first and second transversely spaced surfaces, wherein the retention element is comprised of at least two material layers,
a plurality of housings each comprising a component of or containing one of the single-use medical device and having an access opening through the housing, and
a lid removably sealed to each housing over the access opening,
wherein the plurality of housings are individually detachably attached to and laterally extend from at least one of the first and second laterally spaced edges in a longitudinally spaced arrangement,
and further wherein each housing is individually detachably attached to the retention element by at least one of:
positioning a minor portion of each housing between the two material lavers and securing this minor portion of each housing to the retention implement, and
positioning a minor portion of each lid between the two material lavers and securing this minor portion of each lid to the retention implement.

2. The strip dispenser of claim 1 wherein (i) the retention implement has a spine portion and a plurality of tabs extending laterally from the spine portion, (ii) the tabs comprise the lids, and (iii) a line of demarcation constituting a line of weakness is provided between each tab and the spine for facilitating individual detachment of each tab with associated housing from the spine.

3. The strip dispenser of claim 1 further comprising an orifice through the retention implement operable for suspending the strip dispenser from a point of attachment on an IV pole.

4. (canceled)

5. The strip dispenser of claim 1 wherein the housings each contain the same single-use medical device and the single-use medical device is a medical end-site disinfecting cap.

6.-7. (canceled)

8. The strip dispenser of claim 1 wherein the retention implement is a flexible film.

9.-10. (canceled)

11. The strip dispenser of claim 1 comprising at least three housings laterally extending from each of the first and second laterally spaced edges.

12. The strip dispenser of claim 1 wherein each of the plurality of housings are directly detachably attached to the retention implement.

13. The strip dispenser of claim 1 wherein the lid on each of the plurality of housings is directly detachably attached to the retention implement.

14. The strip dispenser of claim 1 wherein each of the plurality of housings is detachably attached to the retention implement at a point of attachment, and the strip dispenser further comprises a line of weakness in the retention implement around each point of attachment for facilitating individual detachment of each housing from the retention implement.

15. (canceled)

16. The strip dispenser of claim 1 wherein each of the plurality of housings has a line of weakness configured and arranged for facilitating individual detachment of each housing from the retention implement without opening the housing so as to expose the contents of the housing to the surrounding environment.

17. (canceled)

18. The strip dispenser of claim 1 wherein each of the plurality of housings has a line of weakness configured and arranged for facilitating individual detachment of each housing from the retention implement and opening the housing so as to provide access to the contents of the housing.

19.-21. (canceled)

22. The strip dispenser of claim 1 wherein the bond strength between the lid and the housing exceeds the bond strength between the lid and the retention implement whereby pulling of the housing away from the lid effects detachment of the housing and lid from the retention implement.

23. The strip dispenser of claim 1 wherein the bond strength between the lid and the retention implement exceeds the bond strength between the lid and the housing whereby pulling of the housing away from the lid effects detachment of the housing from the lid.

24. The strip dispenser of claim 1 wherein the at least two material layers are laminated to each other and the minor portion of each housing is between the laminated material layers.

25. The strip dispenser of claim 24 wherein the minor portion of each housing and the portion of the laminated material layers overlapping the minor portion of each housing are embossed with nested transversely projecting relief patterns whereby the nested relief patterns are operable for resisting lateral withdrawal of the minor portion of each housing from between the laminate material layers.

26. (canceled)

27. The strip dispenser of claim 1 wherein the at least two material layers are laminated to each other and the minor portion of each lid is between the laminated material layers.

28. The strip dispenser of claim 27 wherein the minor portion of each lid and the portion of the laminated material layers overlapping the minor portion of each lid are embossed with nested transversely projecting relief patterns whereby the nested relief patterns are operable for resisting lateral withdrawal of the minor portion of each lid from between the laminated material layers.

29. (canceled)

30. The strip dispenser of claim 27 wherein (i) the minor portions of each lid positioned between the laminated material layers along the same edge of the retention implement comprise a single unitary sheet of lidstock wherein the minor portions are interconnected as a unitary whole, and (ii) a line of weakness is configured and arranged in each lid between the minor portion of the lid and that portion sealing the opening in the housing for facilitating individual detachment of each housing from the minor portion of each lid without detaching that portion of the lid sealing the housing from the housing.

31.-38. (canceled)

39. A method of manufacturing a strip dispenser for individually dispensing small single-use medical devices, comprising the steps of:
(a) obtaining a first material layer and a second material layer, each having first and second longitudinally spaced ends, first and second laterally spaced edges and first and second transversely spaced surfaces,
(b) obtaining a plurality of housings, each comprising a component of or containing the same small single-use medical device and having an access opening through the housing sealed with a removable lid,
(c) positioning a plurality of the housings in longitudinally spaced relationship to one another along at least one of the edges of the first material layer with a minor portion of each housing overlapping the first surface of the first material layer,
(d) superimposing the second material layer over the first material layer with the first surface of the second material layer facing the first surface of the first material layer so as to sandwich the minor portion of each housing overlapping the first surface of the first material layer between the first material layer and the second material layer, and
(e) bonding the first and second material layers together to form a retention implement with the minor portion of each housing sandwiched between the first and second material layers detachably secured to the retention implement.

40.-45. (canceled)

46. A method of manufacturing a strip dispenser for individually dispensing small single-use medical devices, comprising the steps of:
(a) obtaining a first material layer and a second material layer, each having first and second longitudinally spaced ends, first and second laterally spaced edges and first and second transversely spaced surfaces,
(b) obtaining a plurality of housings, each comprising a component of or containing the same small single-use medical device and having an access opening through the housing sealed with a removable lid,
(c) positioning a plurality of the housings in longitudinally spaced relationship to one another along at least one of the edges of the first material layer with a minor portion of each lid overlapping the first surface of the first material layer,
(d) superimposing the second material layer over the first material layer with the first surface of the second material layer facing the first surface of the first material layer so as to sandwich the minor portion of each lid overlapping the first surface of the first material layer between the first material layer and the second material layer, and
(e) bonding the first and second material layers together to form a retention implement with the minor portion of each lid sandwiched between the first and second material layers detachably secured to the retention implement.

47.-54. (canceled)
